(19) United States
(12) Patent Application Publication
(10) Pub. No.: US 2023/0166120 A1
(43) Pub. Date: Jun. 1, 2023

(54) DEVICE AND METHOD FOR IMAGE-BASED SUPPORT OF A USER

(71) Applicant: Erbe Elektromedizin GmbH, Tuebingen (DE)

(72) Inventors: Matthias Zenker, Tuebingen (DE); Ovidiu Jurjut, Tuebingen (DE); Yannick Daniel, Balingen (DE)

(73) Assignee: Erbe Elektromedizin GmbH, Tuebingen (DE)

(21) Appl. No.: 17/991,399

(22) Filed: Nov. 21, 2022

(30) Foreign Application Priority Data
Dec. 1, 2021 (EP) 21211739.4

(51) Int. Cl. A61N 1/44; G06T 7/90; G06V 40/10; G06T 7/70

(52) U.S. Cl. CPC A61N 1/44; G06T 7/90; G06V 40/10; G06T 7/70; G06T 2207/10024; G06T 2207/30004

(57) ABSTRACT

Devices and methods for image-based treatment with an instrument configured to apply variable amounts of treatment medium at different locations. Devices having an image caption device that is configured to capture the area to be treated. The device comprises an evaluation device that is configured to determine a treatment trace of the treatment medium relative to the area to be treated from data of the image capture device and to determine a spatially resolved dosage for the area to be treated based on the treatment trace. Devices having a representation device that is configured to display the treatment trace and/or the spatially resolved dosage optically for a user, relative to the tissue. Methods to optically indicate the locations of the area to be treated of the tissue surface that have already been treated and/or the dosage with which the respective treated locations have been treated.

### CROSS-REFERENCE TO RELATED APPLICATION

This application claims priority to European Patent Application No. 21211739.4, filed Dec. 1, 2021, the entirety of which is incorporated by reference herein.

### TECHNICAL FIELD

Embodiments of the invention refer to devices as well as methods for image-based support of the user of a (medical) instrument that is configured to apply a treatment medium on a tissue surface.

### BACKGROUND

Instruments that allow the user to apply a treatment medium, such as a non-thermal plasma, in a handhold manner in a small area on a tissue surface to be treated are known in general. For example, tissue surfaces such as skin, mucosa or wounds are treated thereby by the user in that a treatment medium, such as cold plasma (non-thermal plasma) or an argon plasma having low energy is applied on the tissue surface.

The treatment of the tissue surface results in no or only a minor thermal effect in the tissue, whereby no coagulation or carbonization of the tissue occurs. The local temperature of the tissue surface remains below the denaturing temperature of proteins (approximately 60-70° C.). The treated tissue surface does not change optically so that the user is unable to recognize in which area of the tissue surface the treatment medium has already been applied. Also the user cannot recognize the dosage and influence duration that individual sites of the area to be treated have received.

Therefore, the user has to memorize as accurately as possible in which areas and during which amount of time the treatment medium has been applied.

This puts an increase demand on the concentration of the user. In addition, the estimation of the time duration during which the treatment medium has been applied to a specific location is subject to the individual perception of the user. In addition, due to the absence of visible effects of the treatment on the tissue the user can tend to move over already treated portions again and again and in doing so to produce an overdosage.

Similarly, it can, however, happen that the user does not treat individual areas at all or only for a short period and therefore these areas are subject to underdosage.

This results in that the treatment quality and the uniformity, which the treatment medium is applied on the tissue surface may vary remarkably from time to time. Therefore also the treatment results are only difficult to reproduce and cannot be checked in an objective manner and/or documented.

Approaches exist in which it is tried to improve the uniformity, which the treatment medium is applied on the tissue surface by newly developed large size plasma sources. For example DE 10 2014 220 488 A1 suggests a large size plasma source for cold plasma under atmospheric pressure by means of which a relatively large area (for example an area of approximately 100 cm²) can be treated. Particularly, in case of areas to be treated that do not have simple shapes (such as rectangular, square, elliptical or circular) but complex and or irregular shapes it can occur that locations are unintentionally treated that are located outside of the area to be treated.

Additional known approaches propose automatization of the application of the treatment medium. For example, DE 10 2010 011 64 A1 proposes a plasma source that is automatically moved by means of a movement device controlled by control device relative the tissue surface. However, this directly influences the treatment process. In addition, a complete automatization of the application requires high development efforts and is thus costly.

In “Concept for Improved Handling Ensures Effective Contactless Plasma Treatment of Patients with kINPen® MED”, Applied Sciences, Bd. 10, Nr. 17, Art. Nr. 17, Jan. 2020, doi: 10.3390/app10176133 a sensor system is proposed that determines the distance of the instruments to the treatment region and illuminates the area in which the plasma is expected to be applied. However, this is indicated to the user only for the present point in time. Therefore, the user has to continue to memorize in which areas the treatment medium has been applied during which amount of time.

In addition WO 2011/044248 A2 discloses a system in which a surgical area is monitored during an ablation process by means of a video camera whereby the quality of the lesions created in the tissue during ablation is evaluated by means of a processor. According to the quality that has been determined color markers are shown in the live images of the operation field.

An assisting device for image based support of a surgeon during a surgical intervention is described in DE 10 2015 100 927 A1. The assistance device comprises a camera and an image processing unit wherein the image processing unit is configured to recognize the used medical instrument without the requirement of markers attached on the instrument.

### SUMMARY

Starting therefrom it is an object to provide devices and methods that allow to indicate to the user of an instrument by means of which a treatment medium can be applied on a tissue surface in a handhold manner, which location of the tissue to be treated has already been treated.

This object is solved by means of a device for image-based support of a user according to claim 1.

A device according to an embodiment of the invention comprises an instrument, an image capture device, an evaluation device and a representation device. The instrument is configured to apply treatment medium on alterable locations inside an area to be treated on a tissue surface. The image capture device is configured to detect the area to be treated. The evaluation device is configured to determine a treatment trace of a treatment medium relative to the area to be treated from the data of the image capture device and to determine a spatially resolved dosage for the area to be treated based on the treatment trace. In addition, the representation device is configured to visually display the treatment trace and/or the spatially resolved dosage with reference to the tissue for the user.

In doing so, it becomes possible to visually indicate to the user, which locations of the area to be treated of the tissue surface have already been treated. It becomes particularly possible to indicate to the user which dosage the respective locations have received. Thus, the user can decide on which location of the area to be treated treatment medium should still be applied in order to achieve the distribution of the treatment medium that is as uniform as possible. The danger of an overdose or underdose of individual locations of the area to be treated can be reduced in this manner.

This can result in that the uniformity with which the treatment medium is applied on the tissue surface can be increased whereby the result of the application can be evaluated and can be reproduced hereby.

A particularity of the device according to an embodiment of the invention is the evaluation device by which the treatment trace of the treatment medium relative to the area to be treated is determined. Thereby it becomes possible to determine a spatially resolved dosage for the area to be treated based on the treatment trace. The treatment trace and/or the spatially resolved dosage are visually represented relative to the area to be treated by the representation device.

Hereby a spatially resolved dosage can be a dosage value that is assigned to a location in the area to be treated. This dosage value can have binary conditions (for example treated or non-treated condition) or can also have continuous conditions (for example percentage value indications relative to a predetermined desired dose). The treatment trace can be a time-dependent progress of the position and/or the size (such as the diameter or the radius) of the application area. The application area is preferably the area on which the treatment medium is applied on the tissue surface by means of the instrument at a present point in time. This area can be elliptical or circular, for example. The evaluation device can preferably be a computer or processor device.

The representation device can comprise a speaker unit by which the spatially resolved dosage can be represented acoustically. For example, a sound with varying characteristic (frequency (tone pitch), loudness or a sound sequence that varies depending on the dosage) can be output for his purpose. The characteristic can depend on whether the treatment medium impacts on tissue that has already been treated or that is still non-treated. In addition, the variation of the sound characteristic can be used to indicate a too long residence time of the treatment medium on the tissue.

The treatment medium that can be applied is a plasma, particularly a low or non-thermal plasma. The plasma preferably creates no macroscopic visible thermal effect on the tissue surface. During the treatment with such a plasma the local temperature of the tissue surface can remain below the denaturation temperature of proteins, that is below a temperature limit of 40 to 70° C., preferably below a temperature of 60 to 70° C. A non-thermal plasma is a plasma that is not in thermal balance, hence, in which the temperatures of the contained particles (ions, electrons or neutral particles) distinguish remarkably. In a low thermal plasma, the plasma is also not in thermal balance, so that the temperatures of particles (ions, electrons or neutral particles) contained in the low thermal plasma distinguish. However, the amount by which the temperatures of the contained particles distinguished by low-thermal plasma is lower compared with a non-thermal plasma. In addition, the applied treatment medium can be a low energy argon plasma, with which no visible change of the tissue results on the treated tissue surface. This is at least the case if the plasma does not impact longer than a maximum duration of, for example, some seconds, on the same location of the tissue.

It is preferred if the image capture device is configured to capture the area to be treated at least partly in an image sequence comprising a multiplicity of individual images. The treatment medium can be detected and traced in the individual images of the image sequence. The image sequence can also be a continuous stream of individual images whereby a current individual image is considered as live image of the detected area.

In a preferred embodiment, the evaluation device is configured to recognize the treatment medium and/or a head of the instrument in the image sequence and preferably trace it over multiple individual images of the image sequence. In doing so, a position of the treatment medium relative to the area to be treated of the tissue surface can be determined. In the event that the treatment medium is obscured by the head of the instrument in the current individual image, the position of the instrument can serve to estimate the position of the treatment medium. For recognition of the head of the instrument image recognition algorithms from the field of machine learning can be used, such as neural networks, support-vector-machines or the like.

Preferably, the evaluation device is configured to determine a position and/or a size of the applied treatment medium in a current individual image of the image sequence. This allows tracing of the treatment medium in the image sequence in a simplest possible and robust manner. For example, the position of the treatment medium can be a center point of the application area in which the treatment medium impacts on the tissue surface represented in the individual image. The application area can be an ellipse or circle, for example. The size of the applied treatment area can thereby be a radius or a diameter or an area of the region in which the treatment medium impacts on the tissue surface.

Particularly, the evaluation device can be configured to determine a position, an orientation and/or a dimension of the head of the instrument in the current individual image of the image sequence. Based on additional information, such as position, orientation and/or a dimension of the head, the position of the treatment medium can be determined or estimated in the event that the instrument obscures the treatment medium.

In a simplified embodiment only the position of the head can be used as measure for the position of the treatment medium. Treatment media can be detected, and their position can be determined, which are difficult to detect, such as faint treatment media and/or treatment media having an impact region on the tissue surface with irregular shape. Thereby, the computing power required for the realization can be reduced.

In a preferred embodiment the evaluation device is configured to recognize the treatment medium in the data of the image capture device based on a color and/or shape feature. Preferably, the evaluation device is configured to recognize the treatment medium based on at least one of the following features: a color value range, a brightness value range, and a saturation value range. By using a color and/or shape feature for recognition of the treatment medium a treatment medium can be detected in a simplest possible and robust manner.

Particularly, the evaluation device can be configured to recognize the head of the instrument in the data of the image capture device based on a color and/or shape feature or based on a marker attached on the head. In doing so, the head of the instrument can be recognized with remarkable simple methods that do not require high computing power, whereby the position of the head relative to the area to be treated can be determined.

Preferably, the evaluation device is configured to recognize the treatment medium in a search area in the current individual image by means of a search routine. The search area for the treatment medium can be varied based on the position, the orientation and/or the dimension of the head. This allows to limit the search area for the treatment medium to a smaller area, for example, that adjoins the tip of the head of the instrument.

In a preferred embodiment the evaluation device is configured to determine a predicted position of the treatment medium in the current individual image based on a position and/or velocity of the treatment medium of a previous individual image.

Preferably, the search area, in which the search routine recognizes the treatment medium in the current individual image, is varied based on the predicted position. For example, a condition estimation for the position (a predicted position) of the treatment medium for the current individual image can be determined by an alpha-beta-filter or a Kalman-filter. The search area of the search routine can be located around the predicted position. The size of the search area can be set to a predefined value, for example. Alternatively, the size of the search area can be varied based on the predicted position and/or the velocity and/or a covariance value for the predicted position. The search area can be reduced in case of a high covariance value and can be enlarged conversely in case of a low covariance value. In doing so, tracing of the treatment medium can be improved.

Particularly, the search area defined in the current individual image based on the predicted position of the treatment medium can be used to eliminate wrong positive mismatches. The search routine can continuously search for the treatment medium in the entire current individual image. However, detections that are located outside the search area can be eliminated as wrong positive mismatches. The eliminated wrong positive mismatches can be directly dismissed, for example. In doing so, wrong positive mismatches can be reduced.

In addition or as an alternative, the evaluation device can be configured so that the area to be treated can be defined prior to the application of the treatment medium by the user. For example, the user can retrace the area to be treated in the field of view of the image capture device by means of the instruments without applying treatment medium prior to the start of the application. The evaluation device can be configured to recognize and trace the head of the instrument in order to define the desired area to be treated in this manner. The area to be treated defined in this manner can have an arbitrary shape of arbitrary complexity because it can be input freehand by the user. Alternatively, the definition of the area to be treated can also be carried out by the user, for example on a touch screen. If now the user applies treatment medium onto the tissue surface specific detections of the treatment medium that are located outside the area to be treated can be eliminated as wrong positive mismatch. This can allow reduction of wrong positive mismatches that can be coupled in, for example by a light reflection on metallic clamps outside the area to be treated.

Preferably, the evaluation device is configured to recognize characteristic image features of the area to be treated or reference markers applied in the area of the tissue surface in the image capture device and to assign the treatment trace spatially accurately to the area to be treated. For recognition of the characteristic image features image recognition algorithms of the field of machine learning can also be used, such as neural networks, particularly convolutional neural networks, support vector machines or the like. For example, the reference marker can be a colored point or a point having a specifically well detectable pattern that is arranged in the area to be treated.

In a preferred embodiment the representation device comprises (augmented reality, AR) glasses configured to project the treatment trace and/or the spatially resolved dosage of the area to be treated spatially accurately in the field of view of the glasses.

In another preferred embodiment the evaluation device is communicatively connected with a supply unit of the instrument, wherein the evaluation device is configured to control the application of the treatment medium by the instrument depending on the local dosage. In doing so, an overdose of individual ranges can be automatically avoided.

Preferably, the evaluation device is communicatively connected with the supply device of the instrument, whereby the evaluation device is configured to determine the spatially resolved dosage in addition based on measurement information of the supply unit. The measurement information can be transmitted from the supply unit to the evaluation device whereby the determination of the spatially resolved dosage can be carried out more precisely.

In a specific embodiment the instrument can be part of a treatment device that comprises the instrument and the supply unit that supplies the instrument with operating means. Operating means can be, for example, fluids such as gases or liquids and/or an electrical voltage, particularly radio frequency alternating voltage. For this, the supply unit can comprise a controllable (radio frequency, RF) generator. The generator can provide the power that the instrument requires for application of the treatment medium. In addition, the generator can comprise a control unit that is, for example, communicatively connected with the evaluation device. The communication between the control unit and the evaluation device can be bidirectional. For example, information about the treatment trace and/or the spatially resolved dosage and/or measurement information, such as an ignition recognition, a current, a voltage, a power and/or resistance can be transmitted from the control unit to the evaluation device. Similarly, other components of the supply unit, for example a gas supply, can be provided with a control unit and can be communicatively connected with the evaluation device, for example, in order to exchange and/or influence data about the gas flow or gas pressure. The components of the supply unit can thereby be an apparatus that can be operated without the presence of the respectively other components.

Preferably, the evaluation unit is configured to separate the area to be treated in a multiplicity of resolution elements for each of which a dosage is determined based on the treatment trace. A resolution element can be a pixel, for example.

In addition, the object is solved by means of a method for image-based support of the user according to claim 15:

Methods described herein comprise the detection of an area to be treated in which a treatment medium can be applied at alterable locations by an instrument. In addition, the methods comprise a determination of a treatment trace of a treatment medium relative to the area. Based on the treatment trace, a spatially resolved dosage for the area to be treated is determined. In addition, the methods comprise the representation of the treatment trace and/or the spatially resolved dosage with reference to the area to be treated.

All of the features and advantages that have been described with reference to devices according to embodiments of the invention can also be used in the methods according described herein.

### BRIEF DESCRIPTION OF THE DRAWINGS

Additional details of advantageous embodiments of the invention are apparent from the drawings, the description as well as the claims. In the drawings show:

FIG. 1 is a schematic view of a device for image-based support of a user in accordance with an embodiment of the invention;

FIG. 2 is a schematic view of another device for image-based support of the user in accordance with an embodiment of the invention;

FIG. 3 is a schematic view of another device for image-based support of a user in accordance with an embodiment of the invention′

FIGS. 4a to 4c are examples of an area to be treated with examples of associated live images of this area in which the treatment trace is illustrated;

FIG. 5 is an example for a live image of an area to be treated;

FIG. 6 is a flow diagram of a method for image-based support of a user in accordance with an embodiment of the invention; and

FIG. 7 is another example for a live image of an area to be treated with predicted position for the treatment medium.

### DETAILED DESCRIPTION

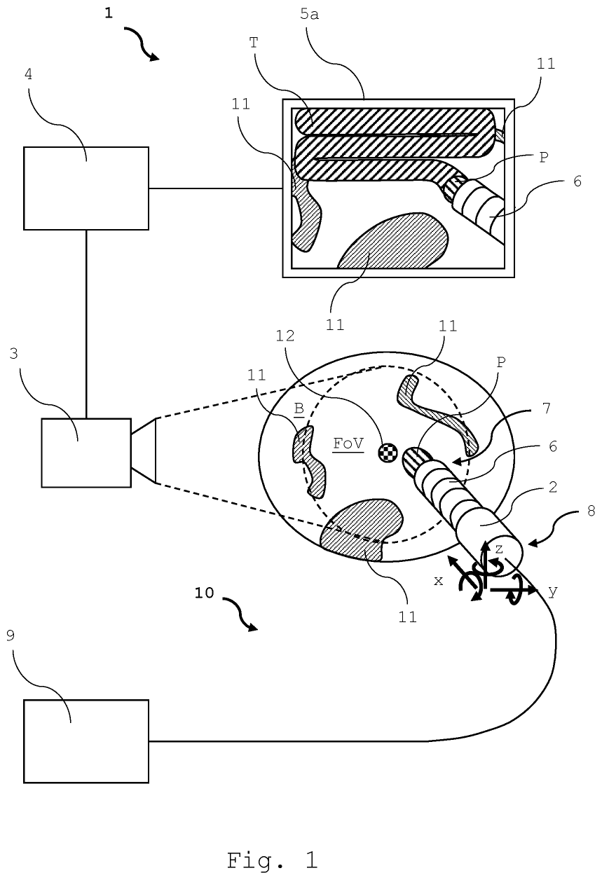

In FIG. 1, a device 1 is illustrated that serves to support a user of a (medical) instrument 2 by means of imaging.

The device 1 comprises an instrument 2 by which a user can apply a treatment medium P in an area to be treated B of a patient. The instrument 2 can be moved freely in the space by the user. The movement of the instrument can follow practically arbitrary paths in the space x, y, z and can thereby also be pivoted in arbitrary directions. By moving and/or rotating the instrument 2, the user can vary the location at which the treatment medium P impacts onto the tissue surface in an area to be treated B.

At a distal end 7 of instrument 2, the instrument 2 comprises a head 6 for generating a treatment medium, for example a plasma, particularly a non-equilibrium plasma (cold plasma). The plasma exits the distal end 7 of instrument 2 and impacts on the tissue surface. The distal end 7 of instrument 2 is the end which is closer to the tissue surface when a treatment medium is applied. Conversely, the proximal end 8 of instrument 2 is the end that is farther away from the tissue surface when a treatment medium is applied. The tissue surface can be, for example, skin, mucosa or wounds in the skin or mucosa. The instrument 2 is supplied by a supply unit 9 comprising an RF generator with electrical power and a suitable gas, for example argon.

The area to be treated B is captured in this embodiment by means of an image capture device 3 at least partly in a field of view (FoV) of the image capture device 3. For example, the image capture device can be a (video) camera. Preferably, the image capture device 3 can be configured to create an image sequence comprising many individual images, wherein the image capture device 3 can also produce a current individual image (live image) of the area to be treated B. The image capture device 3 can be arranged immovably or moveably (handheld) relative to the area to be treated B. If the image capture device 3 is movably arranged relative to the area B, the field of view FoV can move relative to the area B during the treatment.

The image capture device 3 is communicatively connected with an evaluation device 4. The evaluation device 4 is configured to receive and evaluate data from the image capture device 3. In the evaluation device 4, a treatment trace T of the treatment medium P relative to the area B to be treated can be determined from data of the image capture device 3. Based on the treatment trace T a spatially resolved dosage D for the area to be treated can then be determined in the evaluation device 4. If the image capture device 3 is arranged movably relative to the area B, in the evaluation device 4, for example, characteristic features 11 of the area B to be treated can be recognized in the individual images of the image sequence and the movements of the image capture device 3 can be compensated.

The evaluation device 4 is in addition communicatively connected with a representation device. In this embodiment, the representation device 5 is configured as display device 5a, for example as a screen. In the present example, the representation device 5 is configured to represent the treatment trace T for the user optically virtually on the image of the tissue. The representation device 5 can modify the transparency or color intensity of the shown treatment trace T according to the local dosage D. Thus, the influence of the medium on the tissue that is per se traceless is made visible in form of a trace.

In FIG. 1, the treatment trace is shown in a live image of the area to be treated spatially accurately. In the live image in FIG. 1, the characteristic features 11 are also shown. The evaluation device 4 is in addition configured to recognize the characteristic features 11 in the individual images of the image sequence and to calculate the treatment trace T relative to the position of the characteristic features for a current individual image (live image) of the image sequence and to show it therein.

In addition or as an alternative, a reference marker 12 can be adhesively attached on the tissue surface in the area B to be treated. For example, the reference marker 12 can have an optical pattern that can be remarkably easily recognized by the evaluation device 4. In a preferred embodiment, two or more reference markers are adhesively attached onto the tissue surface in order to allow an unambiguous determination of the spatial orientation of each individual image.

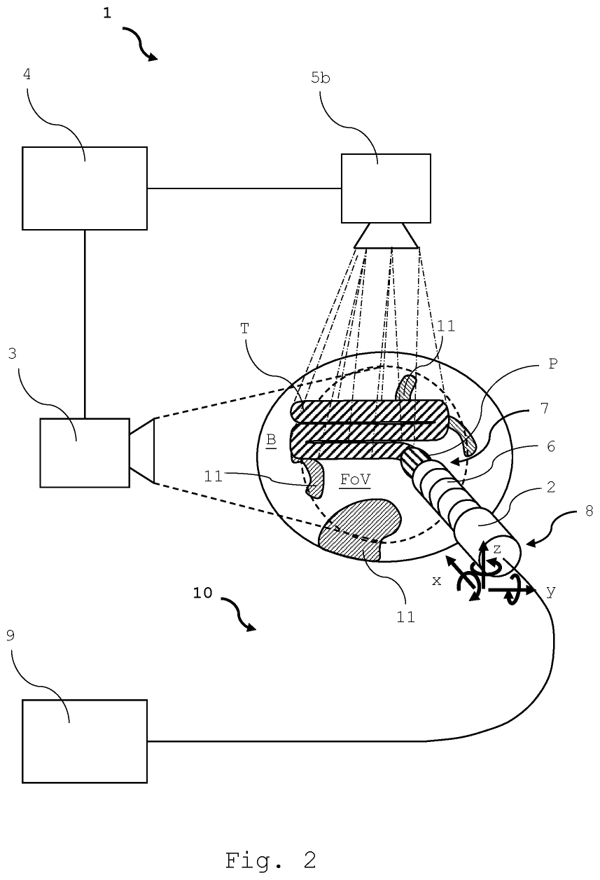

In FIG. 2, a second device in accordance with an embodiment of the invention is illustrated. Instead of the display device 5a, in this example the treatment trace T and the spatially resolved dosage is directly projected into the area B to be treated by means of a projection device 5b. For example, the projection device 5b can comprise a laser or light emitting diode as light source as well as additional optical components such as lenses and mirrors in order to illustrate the treatment trace T respectively.

Alternatively or additionally, the representation device 5 can also comprise augmented reality (AR) glasses 5c that can also be communicatively connected with the evaluation device 4, for example in a wired or wireless manner. The AR glasses 5c are configured to project the spatially resolved dosage of the area to be treated in a spatially accurate manner into the field of view of the glasses.

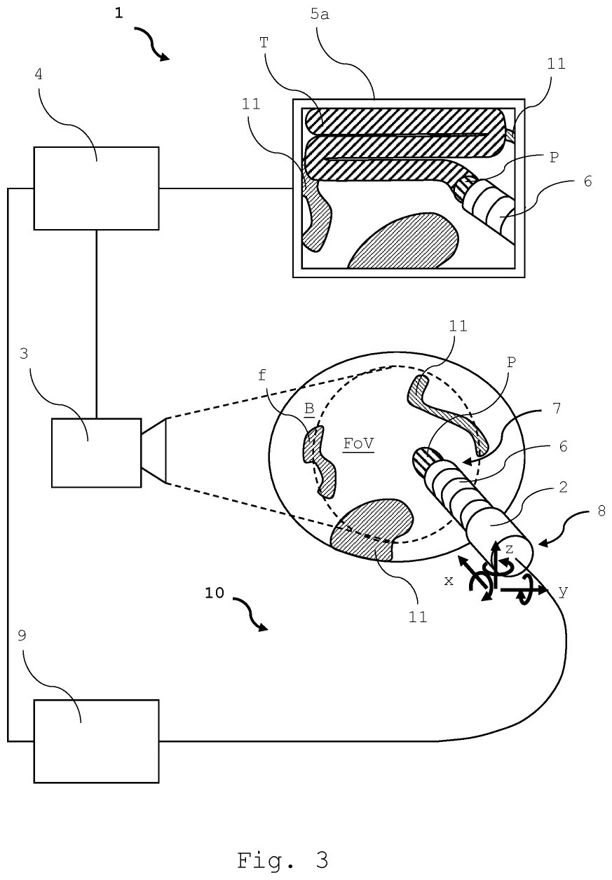

A third device in accordance with an embodiment of the invention is illustrated in FIG. 3. The example of FIG. 3 corresponds substantially to the embodiment of FIG. 1 with the difference that the evaluation device 4 is communicatively connected with the supply unit 9 comprising, amongst others, an RF generator as well as a gas supply and supplies instrument 2.

Due to this communicative connection, the evaluation device 4 can access measurement and control data of the supply unit 9 and use them for the calculation of the dosage. For example, the measurement or control data can be the ignition detection, the power, the output power, the current, the voltage or the resistance for the RF generator or in case of the gas supply unit, the gas flow or the gas pressure.

In a possible alternative embodiment, values of ideal dosage and/or limit values for a minimum and/or a maximum dosage of the tissue can be stored in the evaluation device. The values can either be preset in a non-variable manner or defined by an appropriate input device by the user manually, for example adapted to the patient or the intervention that has to be carried out.

The evaluation device 4 can be configured in this embodiment to vary the application of the treatment medium P depending on the determined local dosage for the region in which the head 6 of instrument 2 is currently located. In this manner, for example, the application of the treatment medium by the instrument can be reduced if the currently treated region is already close to the stored, desired or maximum dosage, that is, if already 70% or 80% or 90% of the desired or maximum dosage are achieved, for example. The application of the treatment medium can also be completely switched off, if the area to be treated has already been treated with the desired or maximum dosage. If the head of the instrument is subsequently moved over a tissue region where the desired or maximum dosage has not been achieved yet, the application of the treatment medium is again increased or allowed. For example, this can be achieved in that the evaluation device 4 provides information about the treatment trace T or the local dosage D to a control unit of the RF generator in the supply unit 9. It is also possible that the evaluation device 4 sends a signal to the control unit for reducing or increasing the power or also for switching off or switching on the power that is supplied to the instrument 2 by the RF generator of the supply unit 9.

The spatially resolved dosage can be calculated from the residence time of the treatment medium P at the individual locations inside the area B to be treated and the adjusted treatment parameters, such as a treatment mode and/or a treatment power. In a simplified embodiment, also only the residence time can be used for calculation of the spatially resolved dosage, if it can be presumed that a predefined treatment mode and/or a predefined treatment power is used for the intervention.

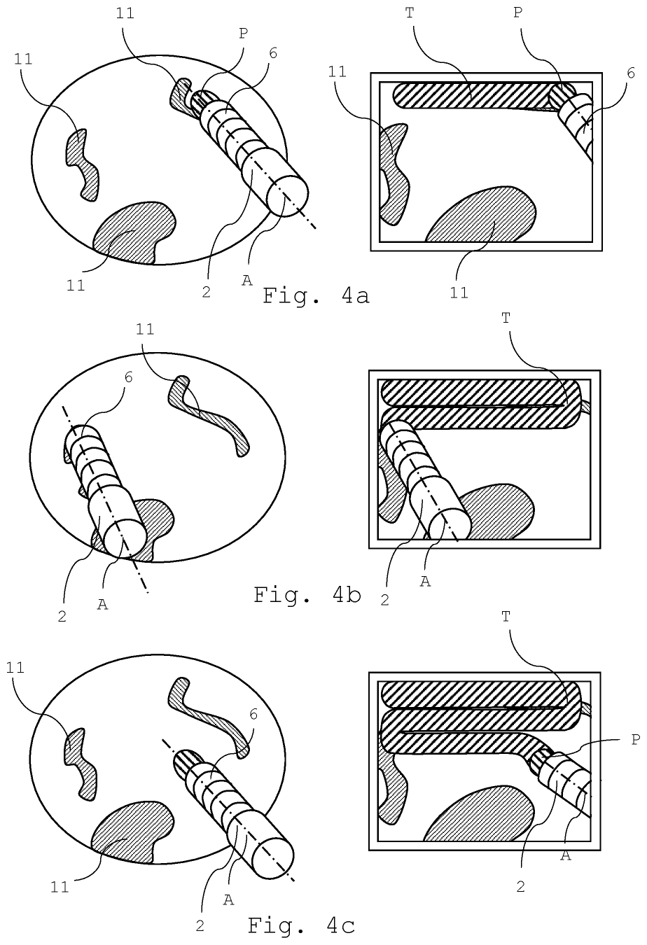

In FIGS. 4a to 4c, an example for an area B to be treated is illustrated together with the associated live image.

On the left side in FIGS. 4a to 4c, the area to be treated B is shown respectively, wherein the plasma P is applied on the tissue surface G by means of the instrument 2. On the right side in FIGS. 4a to 4c, the associated live image is illustrated respectively.

In the live image in FIG. 4a, the current position of the plasma P relative to the area B to be treated has been determined by the evaluation device 4. In addition, in the live image, the treatment trace T is illustrated, that is the time dependent progress of the position of the plasma P inside the area B to be treated.

Alternatively or additionally, instead of the treatment trace T also the dosage D can be shown in the live image. This is for example achieved in that the color or the transparency of the shown treatment trace T in the live image is varied dependent on the local dosage D.

The example of the area B to be treated of FIG. 4a is shown in FIG. 4b. Compared with FIG. 4a, the instrument 2 has been moved further so that the location at which the instrument 2 applies the treatment medium P has been changed. In FIG. 4b, the treatment medium P is obscured by head 6 of instrument 2. For the evaluation device 4 it is for this reason not possible to directly find the treatment medium P in the current individual image of the image sequence. In this case, the evaluation device 4 determines the position and the orientation of head 6 of instrument 2 relative to the area B to be treated.

For this purpose, the evaluation device 4 determines, for example, a main axis A of head 6 of the instrument 2 and, based on the main axis A, a distal direction of the head 6 is determined. Based on the position and the orientation of head 6, the evaluation device 4 determines the position of the obscured treatment medium P. In an embodiment, the evaluation device 4 can use the distal end 7 of head 6 of instrument 2 as position of the treatment medium P on the tissue surface.

In another embodiment, the evaluation device can determine the position of the treatment medium P by extrapolation of the position of the head 6 along the orientation of instrument 2 characterized by the main axis A of instrument head 6. In doing so, also in case of obscured treatment medium P in the current individual image, a precise determination of the position of a treatment medium can be achieved.

The area B to be treated of FIGS. 4a and 4b is shown in FIG. 4c, whereby the instrument 2 has been moved by the user so that the current location has been changed at which the instrument applies the treatment medium P.

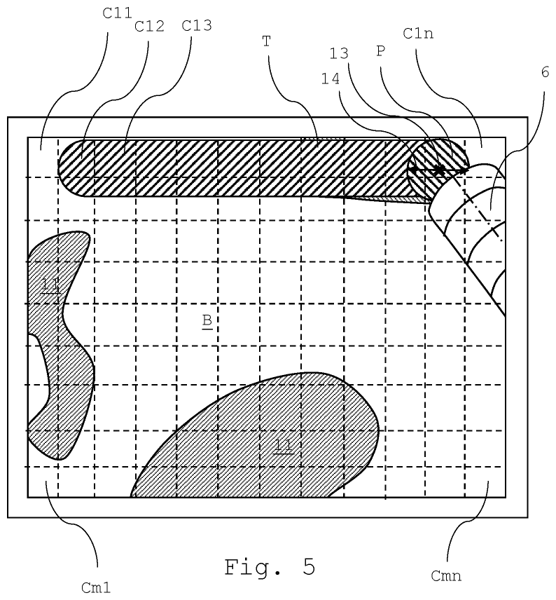

In FIG. 5, an enlarged section of a live image is illustrated schematically. The area B to be treated shown in the live image is separated into a multiplicity of resolution elements C11 to Cmn. For each resolution element a dosage D is determined that is calculated based on the treatment trace T. For example, in FIG. 5 no treatment medium P has been applied in the resolution element C11, because the treatment trace T does not extend through the resolution element C11. Therefore the dosage D11 of resolution element C11 has the value 0. The treatment trace T extends in this example partly through the resolution element C12. For the resolution element C12, the evaluation device 4 can therefore determine a measure for the dosage D12 from the time duration during which the treatment medium P is located inside the resolution element C12 on one hand. On the other hand, the evaluation device 4 can also use the portion of the area of the resolution cell on which the treatment medium P has been applied for the measure. This can be advantageous, particularly for the accuracy of the dosage D12 in an edge region of the treatment medium P. For example, if in the current individual image the position of the treatment medium P is determined as center point of the detected treatment medium P and a border of treatment medium P is placed around the determined center point in the current individual image, the border of treatment medium P can extend between multiple resolution cells so that particularly in the edge region of some resolution cells, an area portion is treated by the treatment medium P. Preferably, a combination of both ways is advantageous, in which the time dependent residence time of the treatment medium P in the resolution element as well as the treated area portion is considered. In FIG. 5, the evaluation device 4 can determine the current position 13 of the treatment medium P as center point of the at least substantially circular region, for example, in which the treatment medium P is currently applied. Also, for example, the evaluation device 4 can determine the diameter 14 of this range as size of the currently applied treatment medium.

Also, the evaluation device 4 can use measurement values of the control of instrument 2, for example an ignition recognition, a current, a voltage, a power or a resistance, to improve the accuracy of the position progress of the treatment medium P (treatment trace T) and the dosage determined therefrom. A relation between the measurement values and the treatment trace can be obtained by means of time information.

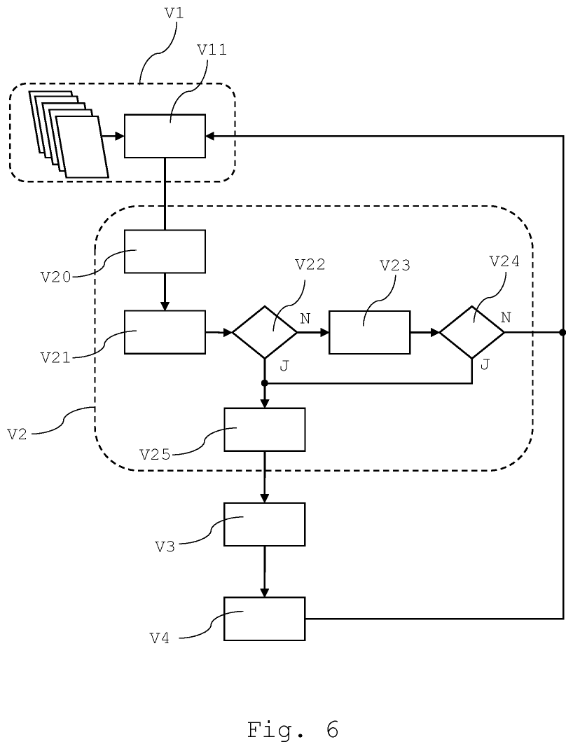

In FIG. 6, a flow diagram of an example of a method for image-based support of a user according to the invention carried out by the evaluation device 4 and the other components of device 2 is shown.

In method step V1, an area B to be treated is captured, inside which treatment medium P can be applied at manually alterable locations. For example, the area B can be captured in an image sequence comprising a multiplicity of individual images. For the further processing steps of the trace loop (tracking loop), a current individual image is now used, for example (method step V11).

In method step V2, a treatment trace T of treatment medium P relative to the area B can be determined. For this purpose, it can be first determined in a method step V20, whether the field of view FoV of the image capture device 3 has been moved relative to the area B in the current individual image. A movement of the field of view FoV can occur, for example, due to a movement, such as a translation and/or rotation, of the image capture device 3 relative to the tissue surface or vice versa. The movement can be determined by comparison of the position and location of characteristic features 11 in the current individual image with the position and location of the characteristic features 11 in a previous individual image. In this manner, occurring movements can be determined quantitatively and can be compensated. Subsequently, the treatment medium P can be searched in the current individual image and position 13 and/or the size 14 of the currently applied treatment medium P can be determined (method step V21). If the treatment medium P cannot be found in the current individual image (query in method step V22) the head 6 of instrument 2 can be searched and the position, the orientation and/or the size of head 6 can be determined (method step V23).

If the treatment medium P has been found in the current individual image, that is a current position 13 and/or size 14 of treatment medium P has been determined for the current individual image, the treatment trace T can be determined or updated in method step V25. For this purpose, for example, the determined, current position 13 as well as the size of the treatment medium P can be stored for each respective individual image so that a treatment trace T results in the image sequence from the positions 13 and sizes 14 of the individual images as a whole. The spatially resolved dosage D can now be determined by an evaluation of the spatial progress and preferably the time dependent progress of the position and size of the treatment medium (treatment trace) (further on method step V25). If the position of the treatment medium P has not been found directly in a current individual image, but the position of head 6 of instrument 2 has been determined (query in method step V24), the position of the treatment medium P can be determined (estimated) based on the position, the orientation and/or the dimension of head 6 of instrument 2. If a position of head 6 of instrument 2 can neither be found, the method is continued in method step V11, in which now a new current individual image is used as database for the substeps V21 to V25 of method step V2.

It is also possible that the steps V21/V22 and V23/V25 are exchanged in their order. First, the position, the orientation and/or the size of head 6 can be determined. Based on these parameters, a search area in which the search routine searches the treatment medium P in the current individual image can now be varied. Preferably, the search area is now limited to the region adjacent to the distal end 7 of head 6. In doing so, it is no longer necessary to search through the entire current individual image for the treatment medium P.

In addition, it is possible to carry out the method steps V21 and V23 in parallel.

In a simplified embodiment, instead of the position of the treatment medium P (substeps V21 and V22), also only the position of head 6 (substep V23) can be used as measure for the position of the treatment medium P. Therefore, in this example, only the substeps V23 to V25 of method step V2 are carried out.

The treatment trace T is preferably updated in method step V25 according to the estimated position of the treatment medium P.

Based on the treatment trace T, a spatially resolved dosage of the area B to be treated can be determined in method step V3.

In method step V4, the treatment trace T and/or the spatially resolved dosage D can now be illustrated in relation to the area B to be treated, in that the treatment trace is shown in the live image.

After method step V4, method step V11 follows again in which a new current individual image is used as data base for the method steps of the trace loop.

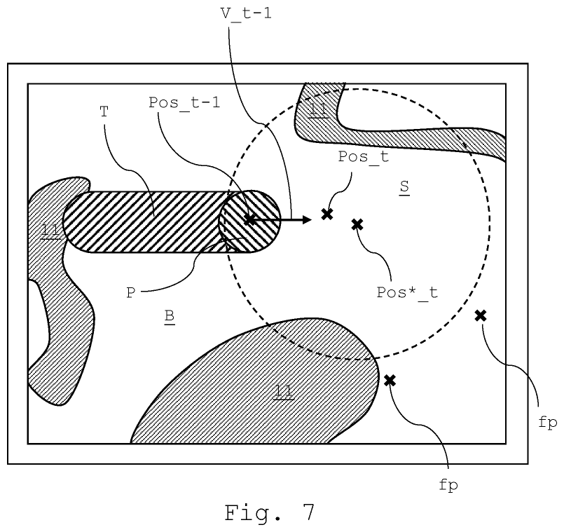

In FIG. 7, an enlarged section of a live image is schematically illustrated. In this embodiment, the evaluation device is configured to determine a predicted position Pos*_t of treatment medium P for the current individual image. The predicted position Pos*_t can be determined based on the previous position Pos*_t−1 and/or the previous velocity V_t−1 of the treatment medium P. In this example, the search area S in which the search routine recognizes the treatment medium in the current individual image is defined around the predicted position Pos*_t. The position Pos_t of treatment medium P detected in the current individual image is located inside the search area S.

Alternatively, the defined search area S can also be used to eliminate wrong positive mismatches of detections fp in which treatment medium P is detected outside the search area S, for example due to light reflections.

In addition, the possibility exists that the size of the search area S is adapted depending on the velocity V_t−1. If the velocity V_t−1 is high, the search area S is made larger. However, if the velocity V_t−1 is low, the search area S can also be smaller.

An embodiment of the operation of the device 1 for image-based support follows:

For carrying out a treatment, the user can apply plasma with the instrument 2, for example a plasma applicator, in a handheld manner in an area to be treated on a tissue surface in a desired dosage. During the treatment, the user can, for example, look at the display device 5a, on which the area to be treated of the tissue surface is shown and/or can wear AR glasses 5c. During the treatment, in addition, the treatment trace T and/or the dosage D can be projected in the area to be treated onto the tissue surface in a manner visible for the user.

While the treatment is carried out it is now indicated to the user which locations of the area to be treated have already been treated with plasma. Preferably the dosage that the individual locations inside the area to be treated have been received is in addition indicated to the user. The user is therefore enabled to apply plasma on non-treated locations or locations with a too low dosage.

For example, in case a treatment of a cervical intraepitheleal neoplasia (CIN), an area of the mucosa surface to be treated is monitored by a video coloscope. In this case, the evaluation device can be connected with the usually already present video coloscope so that the evaluation device can evaluate the (video)data of the video coloscope. Similarly, in other interventions, the camera of an endoscope or laparoscope can be used, for example.

The treatment of a CIN can be carried out, for example, in that a non-thermal plasma is applied onto the mucosa surface. The data of the video coloscope can now be used by the evaluation device in order to determine the time dependent progress of the position of the plasma (treatment trace) and therefrom the spatially resolved dosage. The treatment trace or spatially resolved dosage can accordingly be displayed on a screen of the video coloscope and/or on other screens.

The device 1 according to an embodiment of the invention serves for image-based support of an instrument 2. The instrument 2 is configured to apply a treatment medium at alterable locations in an area to be treated of a tissue surface. In addition, the device comprises an image capture device that is configured to capture the area to be treated. The device 1 comprises an evaluation device 4 that is configured to determine a treatment trace of the treatment medium relative to the area to be treated from data of the image capture device and to determine a spatially resolved dosage for the area to be treated based on the treatment trace. The device 1 also comprises a representation device 5 that is configured to display the treatment trace and/or the spatially resolved dosage optically for the user, relative to the tissue. With the device 1 according to the invention, it is possible to optically indicate to the user which locations of the area to be treated of the tissue surface have already been treated and/or the dosage with which the respective treated locations have been treated.

1. A device for image-based support of a user comprising:
an instrument that is configured to apply a treatment medium at alterable locations in an area to be treated;
an image capture device that is configured to capture the area to be treated;
an evaluation device that is configured to determine a treatment trace of the treatment medium relative to the area to be treated from data of the image capture device and to determine a spatially resolved dosage for the area to be treated based on the treatment trace; and
a representation device that is configured to optically display the treatment trace and/or the spatially resolved dosage in relation to the area to be treated for the user.

2. The device according to claim 1, characterized in that the treatment medium that can be applied is a plasma.

3. The device according to claim 1, wherein the image capture device is configured to capture the area to be treated at least partly by an image sequence comprising a multiplicity of individual images.

4. The device according to claim 1, wherein the evaluation device is configured to recognize the treatment medium and/or a head of the instrument in the image sequence and to trace it over multiple individual images in the image sequence.

5. The device according to claim 3, wherein the evaluation device is configured to determine a position and/or a size of the applied treatment medium in a current individual image of the image sequence.

6. The device according to claim 3, wherein the evaluation device is configured to determine a position, an orientation and/or a dimension of the head of the instrument in the current individual image of the image sequence.

7. The device according to claim 1, wherein the evaluation device is configured to recognize the treatment medium in data of the image capture device based on a color and/or shape feature, at least based on one of the following features: a color value range, a brightness value range and a saturation value range.

8. The device according to claim 1, wherein the evaluation device is configured to recognize the head in data of the image capture device based on a color and/or shape feature or based on a marker attached on the head.

9. The device according to claim 4, wherein evaluation device is configured to recognize the treatment medium in a search area in the current individual image by means of a search routine, whereby the search area for the treatment medium is modified based on the position, the orientation and/or the dimension of the head.

10. The device according to claim 9, wherein the evaluation device is configured to determine a predicted position of the treatment medium in the current individual image based on a position and/or velocity of the treatment medium from a previous individual image, wherein preferably the search area in which the search routine recognizes the treatment medium (P) in the current individual image is modified based on the predicted position and/or the velocity.

11. The device according to claim 1, wherein the evaluation device is configured so that the area to be treated can be defined by the user prior to the application of treatment medium.

12. The device according to claim 1, wherein the evaluation device is configured to recognize characteristic image features of the area (B) to be treated in data of the image capture device or to recognize reference markers applied in the area of the tissue surface and to assign the treatment trace to the area to be treated in a spatially accurate manner.

13. The device according to claim 1, wherein the evaluation device is communicatively connected with a supply unit of the instrument, and further wherein the evaluation device is configured to control the application of the treatment medium by the instrument depending on the local dosage.

14. The device according to claim 1, wherein the evaluation device is communicatively connected with a supply unit of the instrument, whereby the evaluation device is configured to determine the spatially resolved dosage in addition based on measurement parameters that are transmitted by the supply unit to the evaluation device.

15. A method for image-based support of a user, comprising:
capturing an area to be treated in which a treatment medium can be applied at alterable locations;
determination of a treatment trace of the treatment medium relative to the area;
determination of a spatially resolved dosage for the area to be treated based on the treatment trace;
representation of the treatment trace and/or the spatially resolved dosage with reference to the area to be treated.
